(19) United States
(12) Patent Application Publication
(10) Pub. No.: US 2023/0172654 A1
(43) Pub. Date: Jun. 8, 2023

(54) END EFFECTOR ASSEMBLY OF A SURGICAL INSTRUMENT AND SURGICAL INSTRUMENT SUCH AS FOR USE IN SURGICAL ROBOTIC SYSTEMS

(71) Applicant: Covidien LP, Mansfield, MA (US)

(72) Inventors: Dylan R. Kingsley, Broomfield, CO (US); Christopher T. Tschudy, Arvada, CO (US); Jason G. Weihe, Longmont, CO (US); Zachary S. Heiliger, Nederland, CO (US); William R. Whitney, Boulder, CO (US); Curtis M. Siebenaller, Frederick, CO (US); Haralambos P. Apostolopoulos, Westminster, CO (US); Russell W. Holbrook, Naples, FL (US)

(21) Appl. No.: 18/073,485

(22) Filed: Dec. 1, 2022

Related U.S. Application Data
(60) Provisional application No. 63/286,399, filed on Dec. 6, 2021.

(51) Int. Cl. A61B 18/14

(52) U.S. Cl. CPC A61B 18/1445; A61B 2018/0063

(57) ABSTRACT

A surgical end effector assembly includes a clevis, first and second jaw members, and first and second lead wires. The clevis defines first and second arms spaced-apart relative to one another and extending from a proximal body and a finger extending from the proximal body at least partially between the arms. The finger defines a mouth at a free distal end thereof and a channel in communication with the mouth. The jaw members include proximal flange portions at least partially disposed between the arms, distal body portions extending distally from the clevis, and electrically conductive tissue contacting surfaces. At least one jaw member is movable to grasp tissue. The lead wires are connected to the tissue contacting surfaces and extend proximally from the distal body portions of the first and second jaw members through the mouth and into the channel of the finger of the clevis.

### CROSS-REFERENCE TO RELATED APPLICATION

This application claims the benefit of, and priority to, U.S. Provisional Patent Application No. 63/286,399, filed on Dec. 6, 2021, the entire contents of which are hereby incorporated herein by reference.

### FIELD

This disclosure relates to surgical instruments and, more specifically, to an end effector assembly of a surgical instrument and surgical instrument incorporating the same such as, for example, for use in surgical robotic systems.

### BACKGROUND

Surgical robotic systems are increasingly utilized in various different surgical procedures. Some surgical robotic systems include a console supporting a robotic arm. One or more different surgical instruments may be configured for use with the surgical robotic system and selectively mountable to the robotic arm. The robotic arm provides one or more inputs to the mounted surgical instrument to enable operation of the mounted surgical instrument.

A surgical forceps, one type of instrument capable of being utilized with a robotic surgical system, relies on mechanical action between its jaw members to grasp, clamp, and constrict tissue. Electrosurgical forceps utilize both controlled mechanical clamping action and energy to heat tissue to treat, e.g., coagulate, cauterize, or seal, tissue. Typically, once tissue is treated, the tissue is severed using a cutting element. Accordingly, electrosurgical forceps are designed to incorporate a cutting element to effectively sever treated tissue. Alternatively, energy based, e.g., thermal, electrical, ultrasonic, etc., cutting mechanisms may be implemented.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of this disclosure is an end effector assembly of a surgical instrument including a clevis, first and second jaw members, and first and second lead wires. The clevis defines a proximal body, first and second arms spaced-apart relative to one another and extending distally from the proximal body, and a finger extending distally from the proximal body at least partially between the first and second arms. The finger defines a mouth at a free distal end thereof and a channel extending therethrough in communication with the mouth. The first and second jaw members are supported by the clevis. Each of the first and second jaw members includes a proximal flange portion at least partially disposed between the first and second arms of the clevis, a distal body portion extending distally from the clevis, and an electrically conductive tissue contacting surface. At least one of the first or second jaw members is movable relative to the other of the first or second jaw members and the clevis between a spaced-apart position and an approximated position for grasping tissue between the electrically conductive tissue contacting surfaces. The first and second lead wires are connected to the electrically conductive tissue contacting surfaces of the first and second jaw members, respectively and extend proximally from the distal body portions of the respective first and second jaw members through the mouth and into the channel of the finger of the clevis.

In an aspect of this disclosure, the mouth is proximally-angled and defines a rounded edge.

In another aspect of this disclosure, the proximal flange portions of the first and second jaw members are pivotably connected via a pivot pin disposed between the first and second arms of the clevis. In such aspects, at least one of the first or second lead wires may extend proximally from the distal body portions of the respective first and second jaw members and traverse the pivot pin before entering the mouth.

In still another aspect of this disclosure, the proximal flange portions of the first and second jaw members each include first and second proximal flags spaced-apart relative to one another. The first proximal flags of the proximal flange portions of the first and second jaw members may be disposed on a first side of the finger while the second proximal flags of the proximal flange portions of the first and second jaw members are disposed on a second, opposite side of the finger.

In yet another aspect of this disclosure, the proximal flange portions of the first and second jaw members define a nested configuration wherein the first and second proximal flags of the proximal flange portion of the first jaw member are disposed between the first and second proximal flags of the proximal flange portion of the second jaw member.

In still yet another aspect of this disclosure, the first and second proximal flags of the proximal flange portion of the second jaw member are fixed relative to the first and second arms of the clevis, respectively, to thereby fix the second jaw member relative to the clevis. In such aspects, the first jaw member is movable relative to the second jaw member and the clevis.

In still yet another aspect of this disclosure, the clevis includes a smoothed transition defining an internal overhang between each of the first and second arms and the proximal body.

In another aspect of this disclosure, the proximal body and at least portions of lengths of the first and second arms taper in a proximal to distal direction such that the clevis defines a smooth taper in a lateral dimension in the proximal to distal direction.

In yet another aspect of this disclosure, the clevis defines a maximum of the lateral dimension at the proximal end of the clevis, the maximum corresponding to a lateral dimension of a portion of a shaft jointed to the clevis.

In still another aspect of this disclosure, the clevis defines a minimum of the lateral dimension towards free ends of the first and second arms, the minimum corresponding to a maximum width of the distal body portions of the first and second jaw members.

Another end effector assembly of a surgical instrument provided in accordance with aspects of this disclosure includes a clevis and first and second jaw members. The clevis defines a proximal body, first and second arms spaced-apart relative to one another and extending distally from the proximal body, a floor extending distally from the proximal body between bottom edges of the first and second arms, and first and second cross supports connecting respective free end portions of the first and second arms with a free end portion of the floor. The proximal body, the floor, the first and second arms, and the first and second cross supports cooperate to define and fully surround first and second longitudinally-extending slots disposed between the first arm and the floor and between the second arm and the floor, respectively. The first and second jaw members are supported by the clevis. Each of the first and second jaw members includes a proximal flange portion at least partially disposed between the first and second arms of the clevis, a distal body portion extending distally from the clevis, and an electrically conductive tissue contacting surface. The first jaw member is movable relative to the second jaw member and the clevis between a spaced-apart position and an approximated position for grasping tissue between the electrically conductive tissue contacting surfaces. The proximal flange portion of the first jaw member includes first and second proximal flags spaced-apart relative to one another. The first and second proximal flags extend through the first and second slots and externally of the clevis in at least the spaced-apart position of the first jaw member.

In an aspect of this disclosure, the proximal flange portion of the second jaw member includes first and second proximal flags spaced-apart relative to one another and fixed relative to the first and second arms, respectively, of the clevis. In such aspects, the first and second proximal flags of the first jaw member may be disposed between the first and second proximal flags of the second jaw member.

In another aspect of this disclosure, the first and second arms define locating features on interior sides thereof to facilitate locating and fixing the first and second proximal flags of the second jaw member relative to the first and second arms, respectively.

In still another aspect of this disclosure, a maximum width of each of the first and second slots is within 15%, within 10%, or within 5% of a maximum thickness of the first and second proximal flags.

In yet another aspect of this disclosure, the clevis tapers similarly as described with respect to any of the above aspects.

In still yet another aspect of this disclosure, the clevis includes a smoothed transition defining an internal overhang between each of the first and second arms and the proximal body at upper edges of the first and second arms.

### BRIEF DESCRIPTION OF THE DRAWINGS

The above and other aspects and features of this disclosure will become more apparent in view of the following detailed description when taken in conjunction with the accompanying drawings wherein like reference numerals identify similar or identical elements.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a perspective view of a surgical instrument provided in accordance with the present disclosure configured for mounting on a robotic arm of a surgical robotic system such as the surgical robotic system of FIG. 1;

FIGS. 6A and 6B are front and rear perspective views, respectively, of a proximal portion of the surgical instrument of FIG. 5, with an outer shell removed;

FIG. 7 is a front perspective view of the proximal portion of the surgical instrument of FIG. 5 with the outer shell and additional internal components removed;

FIG. 8 is a top view of another end effector assembly provided in accordance with this disclosure configured for use with the surgical instrument of FIG. 5 or any other suitable surgical instrument;

FIG. 9 is a top, perspective views of the end effector assembly of FIG. 8;

FIG. 10 is a side, perspective view of a proximal portion of the end effector assembly of FIG. 8 with the jaw members disposed in an approximated position;

FIGS. 11A and 11B are first and second side, perspective views, respectively, of the clevis of the end effector assembly of FIG. 8;

FIG. 12 is a side, perspective view of a proximal portion of the end effector assembly of FIG. 8 with the jaw members disposed in a spaced-apart position;

FIG. 13 is a bottom view of the clevis of the end effector assembly of FIG. 8;

FIGS. 14A and 14B are enlarged, first and second side, perspective views, respectively, of an interior portion of the clevis of the end effector assembly of FIG. 8 including the proximal flags of the second jaw member engaged therein; and

FIGS. 15A and 15B are enlarged, first and second side, perspective views, respectively, of another configuration of an interior portion of the clevis of the end effector assembly of FIG. 8 including the proximal flags of the second jaw member engaged therein.

### DETAILED DESCRIPTION

This disclosure provides an end effector assembly of a surgical instrument and surgical instrument including the same. As described in detail below, the end effector assembly and surgical instrument of this disclosure are configured for use with a surgical robotic system, which may include, for example, a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which, in turn, move the robotic arm in response to the movement command. Those skilled in the art will understand that this disclosure, although described in connection with surgical robotic systems, may also be adapted for use with handheld surgical instrument such as, for example, endoscopic surgical instruments and/or open surgical instruments.

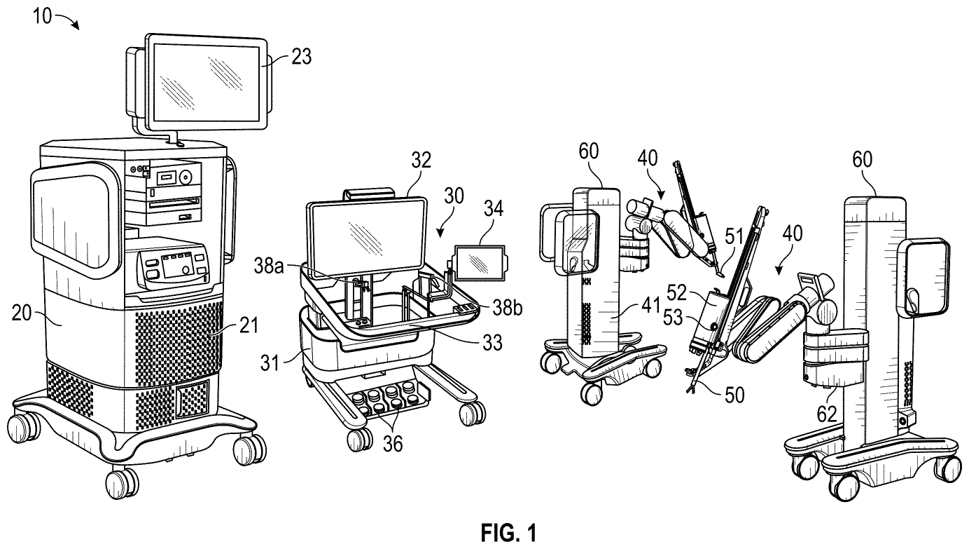

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The one or more surgical instruments 50 may be configured for use during minimally invasive surgical procedures and/or open surgical procedures. In aspects, one of the surgical instruments 50 may be an endoscope, such as an endoscope camera 51, configured to provide a video feed for the clinician. In further aspects, one of the surgical instruments 50 may be an energy based surgical instrument such as, for example, an electrosurgical forceps or ultrasonic sealing and dissection instrument configured to seal tissue by grasping tissue between opposing structures and applying electrosurgical energy or ultrasonic energy, respectively, thereto. In yet further aspects, one of the surgical instruments 50 may be a surgical stapler including a pair of jaws configured to clamp tissue, deploy a plurality of tissue fasteners, e.g., staples, through the clamped tissue, and/or to cut the stapled tissue.

One of the robotic arms 40 may include a camera 51 configured to capture video of the surgical site. The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth® (an open wireless protocol for exchanging data over short distances, using short length radio waves, from fixed and mobile devices, creating personal area networks (PANs)), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

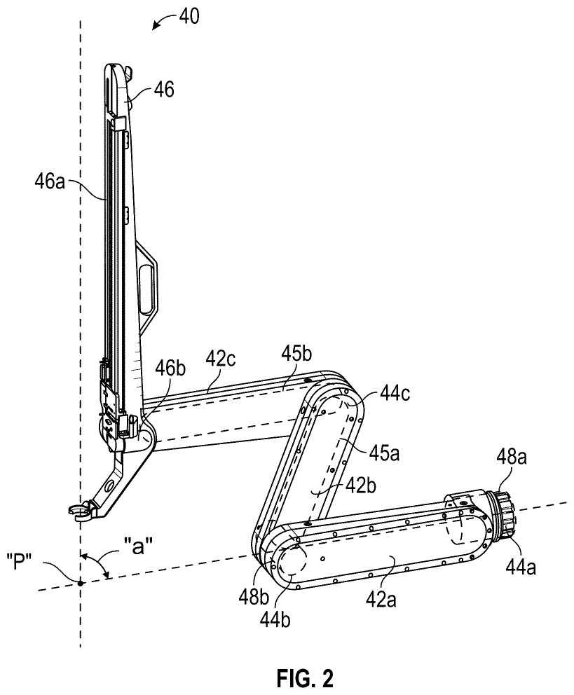

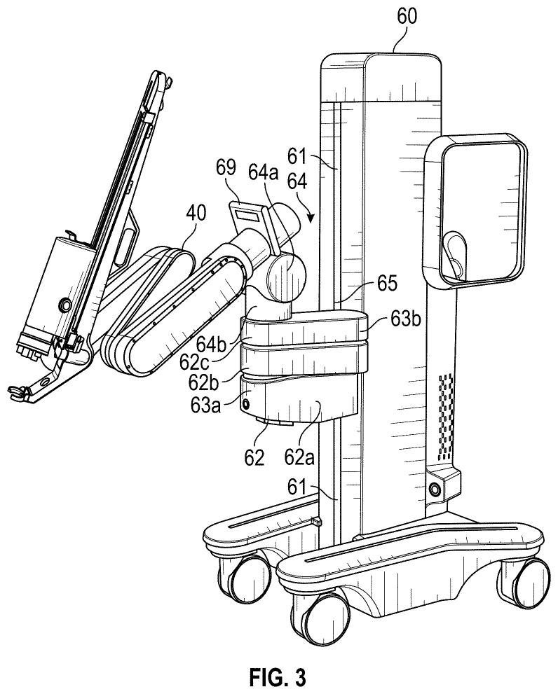

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is traverse to the first stationary arm axis. The first and second actuators 64a and 64b allow for full three-dimensional orientation of the robotic arm 40.

With reference again to FIG. 2, the robotic arm 40 also includes a holder 46 defining a second longitudinal axis and configured to receive an IDU 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 also includes a plurality of manual override buttons 53 disposed on the IDU 52 and the setup arm 62, which may be used in a manual mode. The clinician may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a remote center point “P” which lies at an intersection of the first axis defined by the link 42a and the second axis defined by the holder 46. Thus, the actuator 48b controls the angle “a” between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle “a.” In aspects, some or all of the joints 44a, 44b, 44c may include an actuator to obviate the need for mechanical linkages.

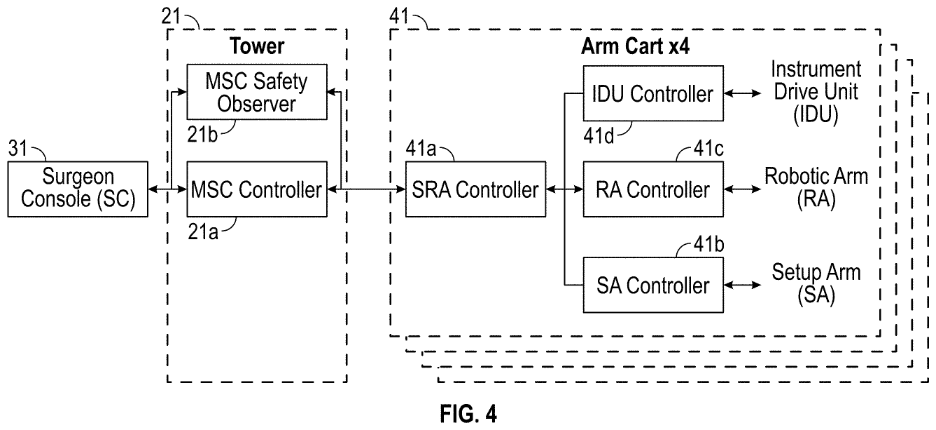

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The handle controllers 38a and 38b include one or more haptic feedback vibratory devices that output haptic feedback. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

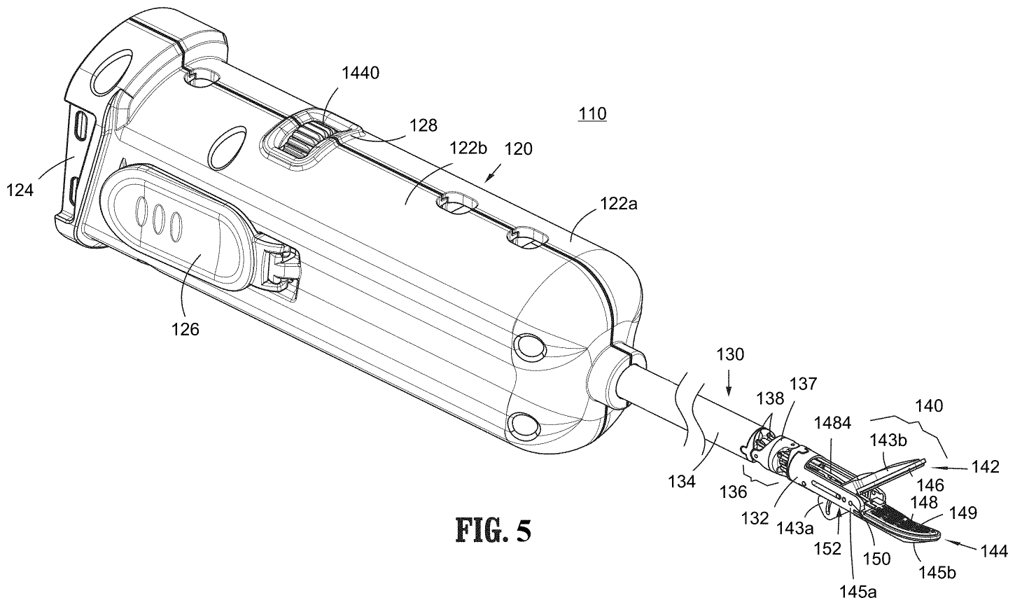

Turning to FIGS. 5-7, a surgical instrument 110 provided in accordance with the present disclosure generally includes a housing 120, a shaft 130 extending distally from housing 120, an end effector assembly 140 extending distally from shaft 130, and an actuation assembly 1100 disposed within housing 120 and operably associated with end effector assembly 140. Instrument 110 is detailed herein as an articulating electrosurgical forceps configured for use with a surgical robotic system, e.g., surgical robotic system 10 (FIG. 1). However, the aspects and features of instrument 110 provided in accordance with the present disclosure, detailed below, are equally applicable for use with other suitable surgical instruments, e.g., graspers, staplers, clip appliers, and/or in other suitable surgical systems, e.g., motorized, other power-driven systems, and/or manually-actuated surgical systems (including handheld instruments).

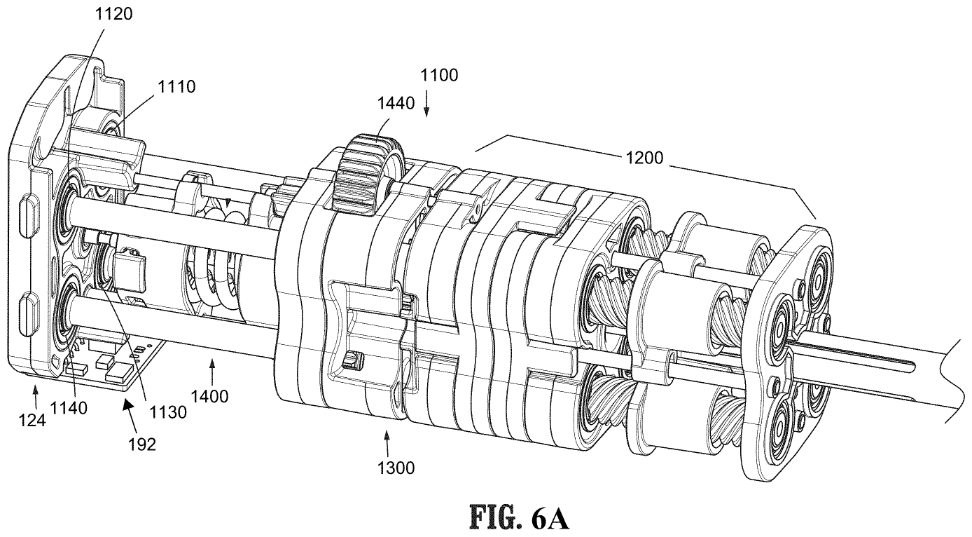

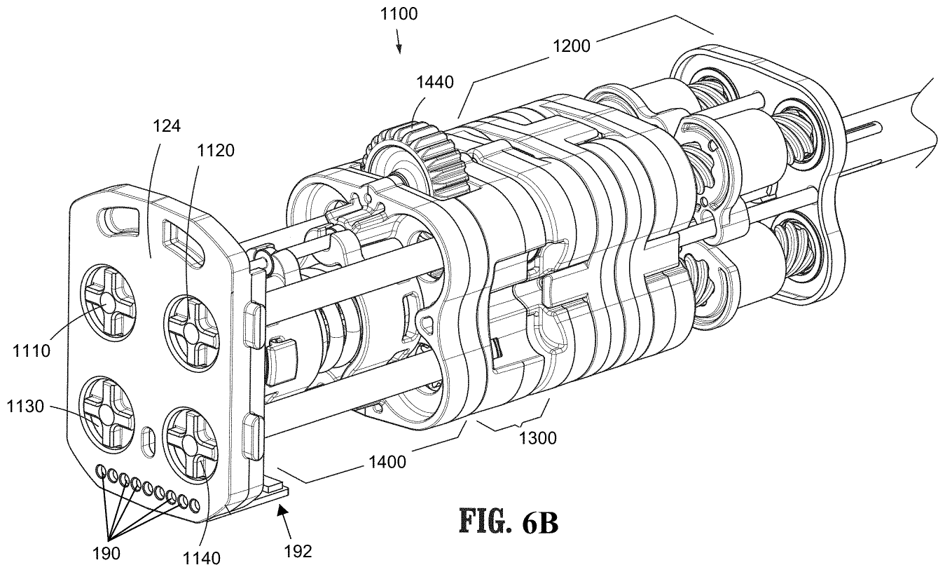

With particular reference to FIG. 5, housing 120 of instrument 110 includes first and second body portion 122a, 122b and a proximal face plate 124 that cooperate to enclose actuation assembly 1100 therein. Proximal face plate 124 includes through holes defined therein through which input couplers 1110-1140 (FIG. 6B) of actuation assembly 1100 extend. A pair of latch levers 126 (only one of which is illustrated in FIG. 5) extending outwardly from opposing sides of housing 120 enable releasable engagement of housing 120 with a robotic arm of a surgical robotic system, e.g., surgical robotic system 10 (FIG. 1). A window 128 defined through housing 120 permits thumbwheel 1440 to extend therethrough to enable manual manipulation of thumbwheel 1440 from the exterior of housing 120 to permit manual opening and closing of end effector assembly 140.

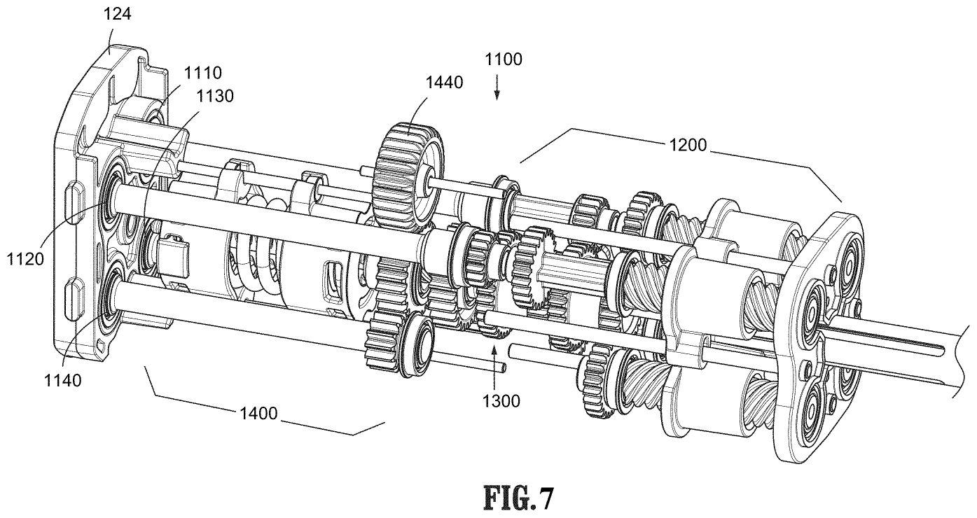

Referring also to FIGS. 6A-7, a plurality of electrical contacts 190 extend through one or more apertures defined through proximal face plate 124 to enable electrical communication between instrument 110 and surgical robotic system 10 (FIG. 1) when instrument 110 is engaged on a robotic arm thereof, e.g., for the communication of data, control, and/or power signals therebetween. As an alternative to electrical contacts 190 extending through proximal face plate 124, other suitable transmitter, receiver, and/or transceiver components to enable the communication of data, control, and/or power signals are also contemplated, e.g., using RFID, Bluetooth®, WiFi®, or via any other suitable wired, wireless, contacted, or contactless communication method. At least some of the electrical contacts 190 are electrically coupled with electronics 192 mounted on an interior side of proximal face plate 124, e.g., within housing 120. Electronics 192 may include, for example, a storage device, a communications device (including suitable input/output components), and a CPU including a memory and a processor. Electronics 192 may be mounted on a circuit board or otherwise configured, e.g., as a chip.

The storage device of electronics 192 stores information relating to surgical instrument such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device of electronics 192 may be, for example, a magnetic disk, flash memory, optical disk, or other suitable data storage device.

As an alternative or in addition to storing the above noted information in the storage device of electronics 192, some or all of such information, e.g., the use information, calibration information, setting information, and/or adjustment information, may be stored in a storage device associated with surgical robotic system 10 (FIG. 1), a remote server, a cloud server, etc., and accessible via instrument 110 and/or surgical robotic system 10 (FIG. 1). In such configurations, the information may, for example, be updated by manufacturer provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer provided updates manually or automatically upon connection to the surgical robotic system 10 (FIG. 1).

Referring again to FIG. 5, shaft 130 of instrument 110 includes a distal segment 132, a proximal segment 134, and an articulating section 136 disposed between the distal and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal segment 132 of shaft 130 at the distal ends thereof and extend proximally from distal segment 132 of shaft 130, through articulating section 136 of shaft 130 and proximal segment 134 of shaft 130, and into housing 120, wherein articulation cables 138 operably couple with an articulation sub-assembly 1200 of actuation assembly 1100 (FIG. 6A) to enable selective articulation of distal segment 132 (and, thus end effector assembly 140) relative to proximal segment 134 and housing 120, e.g., about at least two axes of articulation (yaw and pitch articulation, for example). Articulation cables 138 are arranged in a generally rectangular configuration, although other suitable configurations are also contemplated. In some configurations, as an alternative, shaft 130 is substantially rigid, malleable, or flexible and not configured for active articulation. Articulation sub-assembly 1200 is described in greater detail below.

With respect to articulation of end effector assembly 140 relative to proximal segment 134 of shaft 130, actuation of articulation cables 138 may be accomplished in pairs. More specifically, in order to pitch end effector assembly 140, the upper pair of cables 138 are actuated in a similar manner while the lower pair of cables 138 are actuated in a similar manner relative to one another but an opposite manner relative to the upper pair of cables 138. With respect to yaw articulation, the right pair of cables 138 are actuated in a similar manner while the left pair of cables 138 are actuated in a similar manner relative to one another but an opposite manner relative to the right pair of cables 138. Other configurations of articulation cables 138 or other articulation actuators are also contemplated.

Continuing with reference to FIG. 5, end effector assembly 140 includes first and second jaw members 142, 144, respectively. Each jaw member 142, 144 includes a proximal flange portion 143a, 145a and a distal body portion 143b, 145b, respectively. Distal body portions 143b, 145b define opposed tissue contacting surfaces 146, 148, respectively. Proximal flange portions 143a, 145a are pivotably coupled to one another about a pivot 150 and are operably coupled to one another via a cam slot assembly 152 including a cam pin slidably received within cam slots defined within the proximal flange portion 143a, 145a of at least one of the jaw members 142, 144, respectively, to enable pivoting of jaw member 142 relative to jaw member 144 and distal segment 132 of shaft 130 between a spaced apart position (e.g., an open position of end effector assembly 140) and an approximated position (e.g., a closed position of end effector assembly 140) for grasping tissue between tissue contacting surfaces 146, 148. As an alternative to this unilateral configuration, a bilateral configuration may be provided whereby both jaw members 142, 144 are pivotable relative to one another and distal segment 132 of shaft 130. Other suitable jaw actuation mechanisms are also contemplated.

In configurations, a longitudinally extending knife channel 149 (only knife channel 149 of jaw member 144 is illustrated; the knife channel of jaw member 142 is similarly configured) is defined through the tissue contacting surface 146, 148 of one or both jaw members 142, 144. In such aspects, a knife assembly including a knife rod (not shown) extending from housing 120 through shaft 130 to end effector assembly 140 and a knife (not shown) disposed within end effector assembly 140 between jaw members 142, 144 is provided. The knife is selectively translatable through the knife channel(s) 149 and between the jaw member 142, 144 to cut tissue grasped between tissue contacting surfaces 146, 148 of jaw members 142, 144, respectively. The knife rod is operably coupled to a knife drive sub-assembly 1300 (FIG. 7) of actuation assembly 1100 (FIGS. 6A-6B) at a proximal end thereof to enable the selective actuation of the knife rod to, in turn, reciprocate the knife between jaw members 142, 144 to cut tissue grasped between tissue contacting surfaces 146, 148. As an alternative to a longitudinally advanceable mechanical knife, other suitable mechanical cutters are also contemplated, e.g., guillotine style cutters, as are energy based cutters, e.g., RF electrical cutters, ultrasonic cutters, etc., in static or dynamic configurations.

Referring still to FIG. 5, a drive rod 1484 is operably coupled to cam slot assembly 152 of end effector assembly 140, e.g., engaged with the cam pin thereof, such that longitudinal actuation of drive rod 1484 pivots jaw member 142 relative to jaw member 144 between the spaced apart and approximated positions. More specifically, urging drive rod 1484 proximally pivots jaw member 142 relative to jaw member 144 towards the approximated position while urging drive rod 1484 distally pivots jaw member 142 relative to jaw member 144 towards the spaced apart position. However, other suitable mechanisms and/or configurations for pivoting jaw member 142 relative to jaw member 144 between the spaced apart and approximated positions in response to selective actuation of drive rod 1484 are also contemplated. Drive rod 1484 extends proximally from end effector assembly 140 through shaft 130 and into housing 120 wherein drive rod 1484 is operably coupled with a jaw drive sub-assembly 1400 of actuation assembly 1100 (FIGS. 6A-6B) to enable selective actuation of end effector assembly 140 to grasp tissue therebetween and apply a jaw force within an appropriate jaw force range.

Tissue contacting surfaces 146, 148 of jaw members 142, 144, respectively, are at least partially formed from an electrically conductive material and are energizable to different potentials to enable the conduction of RF electrical energy through tissue grasped therebetween, although tissue contacting surfaces 146, 148 may alternatively be configured to supply any suitable energy, e.g., thermal, microwave, light, ultrasonic, ultrasound, etc., through tissue grasped therebetween for energy based tissue treatment. Instrument 110 defines a conductive pathway (not shown) through housing 120 and shaft 130 to end effector assembly 140 that may include lead wires, contacts, and/or electrically conductive components to enable electrical connection of tissue contacting surfaces 146, 148 of jaw members 142, 144, respectively, to an energy source (not shown), e.g., an electrosurgical generator, for supplying energy to tissue contacting surfaces 146, 148 to treat, e.g., seal, tissue grasped between tissue contacting surfaces 146, 148.

With additional reference to FIGS. 6A-7, as noted above, actuation assembly 1100 is disposed within housing 120 and includes an articulation sub-assembly 1200, a knife drive sub-assembly 1300, and a jaw drive sub-assembly 1400. Articulation sub-assembly 1200 is operably coupled between first and second input couplers 1110, 1120, respectively, of actuation assembly 1100 and articulation cables 138 (FIG. 5) such that, upon receipt of appropriate inputs into first and/or second input couplers 1110, 1120, articulation sub-assembly 1200 manipulates cables 138 (FIG. 5) to articulate end effector assembly 140 in a desired direction, e.g., to pitch and/or yaw end effector assembly 140. Articulation sub-assembly 1200 is described in greater detail below.

Knife drive sub-assembly 1300 is operably coupled between third input coupler 1130 of actuation assembly 1100 and the knife rod such that, upon receipt of appropriate input into third input coupler 1130, knife drive sub-assembly 1300 manipulates the knife rod to reciprocate the knife between jaw members 142, 144 to cut tissue grasped between tissue contacting surfaces 146, 148.

Jaw drive sub-assembly 1400 is operably coupled between fourth input coupler 1140 of actuation assembly 1100 and drive rod 1484 such that, upon receipt of appropriate input into fourth input coupler 1140, jaw drive sub-assembly 1400 pivots jaw members 142, 144 between the spaced apart and approximated positions to grasp tissue therebetween and apply a jaw force within an appropriate jaw force range.

Actuation assembly 1100 is configured to operably interface with a surgical robotic system, e.g., system 10 (FIG. 1), when instrument 110 is mounted on a robotic arm thereof, to enable robotic operation of actuation assembly 1100 to provide the above detailed functionality. That is, surgical robotic system 10 (FIG. 1) selectively provides inputs, e.g., rotational inputs to input couplers 1110-1140 of actuation assembly 1100 to articulate end effector assembly 140, grasp tissue between jaw members 142, 144, and/or cut tissue grasped between jaw members 142, 144. However, as noted above, it is also contemplated that actuation assembly 1100 be configured to interface with any other suitable surgical systems, e.g., a manual surgical handle, a powered surgical handle, etc.

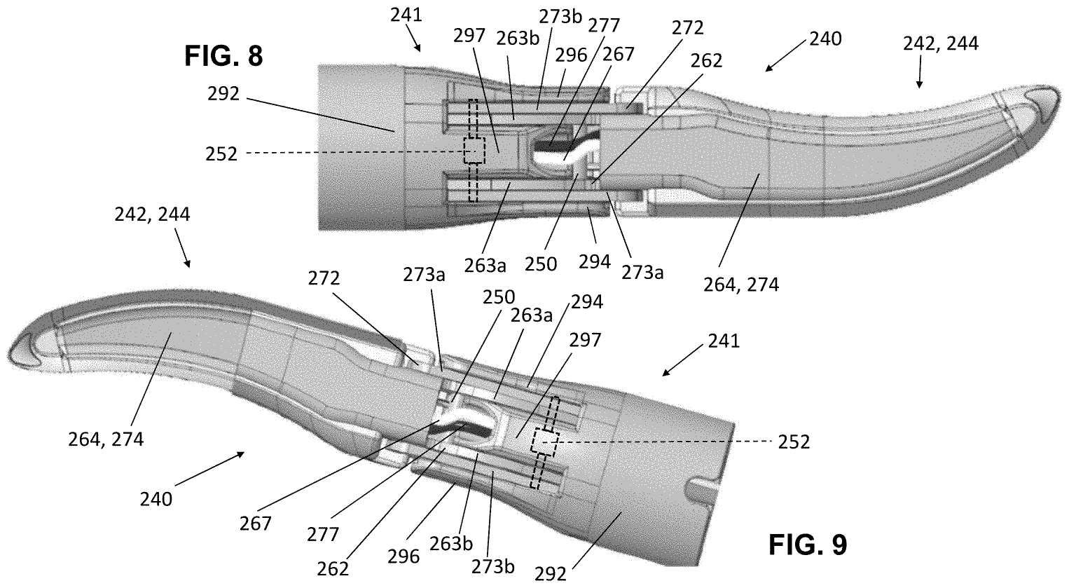

Turning to FIGS. 8-10, another end effector assembly provided in accordance with the present disclosure and configured for use with surgical instrument 110 (FIG. 5) or any other suitable surgical instrument is shown generally identified by reference numeral 240. End effector assembly 240 is similar to and may include any of the features of end effector assembly 140 (FIG. 5), and/or may be utilized as part of surgical instrument 110 (FIGS. 5-7) similarly as described above with respect to end effector assembly 140 (FIG. 5), except as explicitly contradicted below.

End effector assembly 240 includes a clevis 241 joined to or forming distal segment 132 of shaft 130 of instrument 110 (see FIG. 5) and first and second jaw members 242, 244, respectively, operably coupled to clevis 241. More specifically, first jaw member 242 is pivotably coupled to clevis 241 and second jaw member 244, while second jaw member 244 is fixed relative to clevis 241. Each jaw member 242, 244 includes a proximal flange portion 262, 272, and a distal body portion 264, 274 that supports an opposed electrically conductive tissue contacting surface 266, 276, respectively.

Proximal flange portions 262, 272 of jaw members 242, 244 are bifurcated such that each proximal flange portion 262, 272 includes a pair of spaced-apart proximal flags 263a, 263b and 273a, 273b, respectively. Proximal flags 263a, 263b and 273a, 273b are oriented substantially perpendicularly relative to electrically conductive tissue contacting surfaces 266, 276, respectively. Proximal flags 273a, 273b are spaced-apart a greater distance as compared to proximal flags 263a, 263b to allow proximal flags 263a, 263b to be nested between proximal flags 273a, 273b with proximal flags 263a, 273a disposed adjacent if not abutting one another and with proximal flags 263b, 273b disposed adjacent if not abutting one another. As such, a gap is defined between the adjacent or abutting sets of proximal flags 263a, 273a and 263b, 273b.

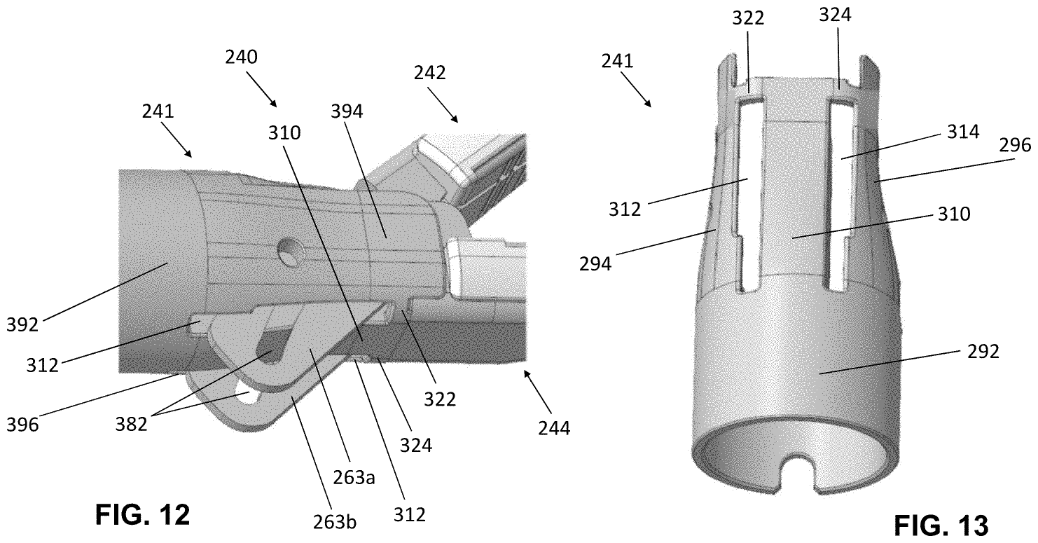

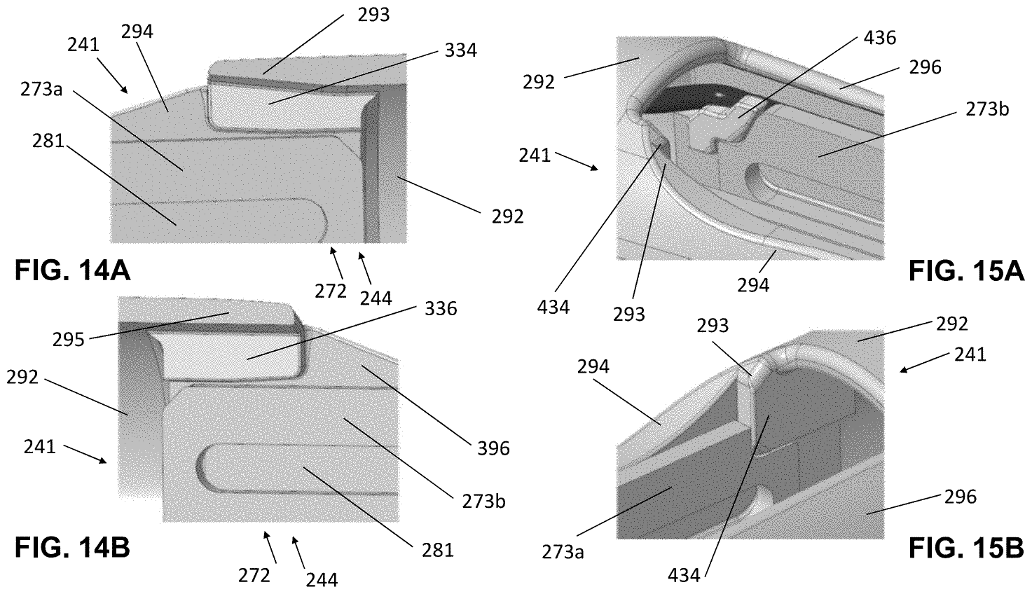

Proximal flags 273a, 273b of jaw member 244 define aligned longitudinally-extending cam slots 281 (FIGS. 14A and 14B) and aligned pivot apertures (not explicitly shown). Proximal flags 263a, 263b of jaw member 242 define aligned arcuate cam slots 382 (FIG. 12) and aligned pivot apertures (not explicitly shown). Upon assembly of proximal flange portions 262, 272 of jaw members 242, 244 within clevis 241, a pivot pin 250 captured between arms 294, 296 of clevis 241 extends through the aligned pairs of pivot apertures (not explicitly shown) to pivotably couple jaw members 242, 244 with one another and to pivotably couple jaw member 242 with clevis 241. As an alternative to capturing pivot pin 250 within clevis 241, pivot pin 250 may extend through arms 294, 296 of clevis 241 in operable engagement therewith; cam driver 252 may similarly be configured. Further, a cam driver 252 including opposing cam bosses or other suitable cam structures extends through arcuate cam slots 382 (FIG. 12) of jaw member 242 and longitudinally-extending cam slots 281 (FIGS. 14A and 14B) of jaw member 244 such that, translation of cam driver 252, e.g., in response to actuation of drive rod 1484 (FIG. 5), moves cam driver 252 through cam slots 281, 382 (FIGS. 12 and 14A-14B, respectively) to thereby pivot jaw member 242 relative to jaw member 244 and clevis 241.

Jaw member 242, more specifically, is pivotable relative to jaw member 244 and clevis 241 between a spaced apart position (e.g., an open position of end effector assembly 240) and an approximated position (e.g., a closed position of end effector assembly 240) for grasping tissue between electrically conductive tissue contacting surfaces 266, 276. As an alternative to this unilateral configuration, a bilateral configuration may be provided whereby both jaw members 242, 244 are pivotable relative to one another and clevis 241. Any suitable jaw actuation mechanism such as, for example, using a push-pull drive bar (e.g., drive bar 1484 (FIG. 5) as detailed above), may be utilized to actuate jaw member(s) 242, 244.

Continuing with reference to FIGS. 8-10, electrically conductive tissue contacting surfaces 266, 276 of jaw members 242, 244, respectively, are at least partially formed from an electrically conductive material and are energizable to different potentials to enable the conduction of RF electrical energy through tissue grasped therebetween, although electrically conductive tissue contacting surfaces 266, 276 may alternatively be configured to supply any suitable energy, e.g., thermal, microwave, light, ultrasonic, ultrasound, etc., through tissue grasped therebetween for energy based tissue treatment. In order to enable the conduction of energy to and from electrically conductive tissue contacting surfaces 266, 276, electrical lead wires 267, 277 electrically connect to electrically conductive tissue contacting surfaces 266, 276 within jaw members 242, 244 and extend proximally therefrom through clevis 241, shaft 130 of instrument 110, and into housing 120 (see FIG. 5) for ultimate direct or indirect connection to an energy source (not shown), e.g., an electrosurgical generator, to enable the supply of energy to electrically conductive tissue contacting surfaces 266, 276 to treat, e.g., seal, tissue grasped therebetween. Electrical lead wires 267, 277 are routed from jaw members 242, 244, with at least one of electrical lead wires 267, 277 traversing pivot pin 250 before extending through clevis 241, as detailed below.

With additional reference to FIGS. 11A and 11B, clevis 241 includes a generally cylindrical proximal body 292 that is joined to or forms distal segment 132 of shaft 130 of instrument 110 (see FIG. 5) and first and second spaced-apart arms 294, 296 that extend distally from proximal body 292. Clevis 241 operably retains proximal flange portions 262, 272 of jaw members 242, 244, pivot pin 250, and cam driver 252 between arms 294, 296 such that clevis 241 and first and second jaw members 242, 244 are operably coupled to one another. More specifically, first jaw member 242 is pivotably coupled to clevis 241 and second jaw member 244 between spaced-apart arms 294, 296, e.g., via pivot pin 250, while second jaw member 244 is fixed relative to clevis 241, e.g., via welding proximal flags 273a, 273b on the interior sides of spaced-apart arms 294, 296, respectively, or via integrally forming clevis 241 and second jaw member 244 as a single component.

As best shown in FIG. 8, clevis 241 defines a tapered outer lateral dimension whereby the proximal end of cylindrical proximal body 292 of clevis 241 defines an outer lateral width that substantially approximates the outer diameter of distal segment 132 of shaft 130 of instrument 110 (see FIG. 5). This corresponds to a maximum width of clevis 241. More specifically, clevis 241 smoothly tapers in width from the proximal end of cylindrical proximal body 292 to a minimum outer lateral dimension towards free ends of first and second spaced-apart arms 294, 296. To provide this smooth taper, first and second spaced-apart arms 294, 296 are tapered in thickness along at least portions of the lengths thereof. The minimum outer lateral dimension of clevis 241 corresponds to the maximum widths of distal body portions 264, 274 of jaw members 242, 244 which are defined at the proximal ends of distal body portions 264, 274. Further, although jaw members 242, 244 define curved configurations, jaw members 242, 244 do not extend laterally (in the direction of curvature thereof) out of a radial envelope defined by the maximum width of clevis 241 at the proximal end of cylindrical proximal body 292. This tapered configuration of clevis 241 together with the configuration of jaw members 242, 244, which also taper in outer lateral dimension, facilitates visualization.

Referring again to FIGS. 8-11B, in addition to the tapered configuration of clevis 241, clevis 241 defines smooth, curved transitions 293, 295 between cylindrical proximal body 292 and arms 294, 296 at the upper (and, in aspects, lower ends) of arms 294, 296. Transitions 293, 295 eliminate or reduce exposed flat edges and angles and also define overhangs that extend into the gap defined between arms 294, 296 to thereby to fill some of that gap. The various features of clevis 241 detailed above and otherwise herein provide a configuration wherein pinch or catch points are substantially eliminated or reduced. Thus, clevis 241 provides a substantially smooth exterior to facilitate atraumatic sliding against tissue and for insertion/removal through a surgical access device, e.g., a cannula.

Clevis 241 further includes a finger 297 extending from cylindrical proximal body 292 laterally between arms 294, 296, although finger 297 may be at least partially offset above arms 294, 296. Finger 297 defines a proximally-angled and smooth-edged (e.g., rounded-edge) mouth 298 that provides access to a lead wire channel 299 extending through finger 297. Lead wire channel 299 may further extend through at least a portion of cylindrical proximal body 292. Lead wire channel 299 is configured to receive electrical lead wires 267, 277 of jaw members 242, 244, respectively which, as noted above, are routed from jaw members 242, 244, at least one of which traverses pivot pin 250 (e.g., from a lower side thereof to an upper side thereof or vice versa), and both of which are routed proximally beyond pivot pin 250, to guide electrical lead wires 267, 277 proximally from end effector assembly 140 into shaft 130 (FIG. 5). With end effector assembly 240 fully assembled, finger 297 is disposed laterally between proximal flags 263a, 263b of jaw member 242.

Turning to FIGS. 12 and 13, as noted above, first jaw member 242 is pivotably and operably coupled to clevis 241 and second jaw member 244 between spaced-apart arms 294, 296. In order to enable first jaw member 242 to pivot relative to second jaw members 244 and clevis 241, e.g., to the fully spaced-apart position, the proximal end portions of proximal flags 263a, 263b extend below and outside of clevis 241. Thus, clevis 241 must include one or more openings to enable proximal flags 263a, 263b to extend outwardly from clevis 241. However, this must be balanced with minimizing openings which may increase the incidence of tissue, fluid, and debris ingress and/or create catch or pinch points. Clevis 241 thus includes a floor 310 extending from cylindrical proximal body 292 laterally between arms 294, 296 at the bottoms of arms 294, 296. Floor 310 has a width less than the distance between arms 294, 296 such that first and second slots 312, 314 are defined between floor 310 and arm 294 and between floor 310 and arm 296, respectively. Slots 312, 314 are configured to receive proximal flags 263a, 263b to enable first jaw member 242 to pivot relative to second jaw members 244 and clevis 241, e.g., to the fully spaced-apart position. Slots 312, 314 define maximum widths that are only slightly greater, e.g., less than 15% greater, less than 10% greater, or less than 5% greater, than the maximum thicknesses of proximal flags 263a, 263b, thus defining minimal open space therebetween.

Clevis 241 further includes cross supports 322, 324 extending traversely between arm 294 and floor 310 and arm 296 and floor 310, respectively, at the free end of floor 310 to thereby fully enclose slots 312, 314 on all sides. By connecting arms 294, 296 with floor 310, cross supports 322, 324 not only eliminate open space to inhibit ingress and/or catch or pinch points, but cross supports 322, 324 also provide structural reinforcement to arms 294, 296 towards the free ends thereof, thus inhibiting splay or rotation of arms 294, 296.

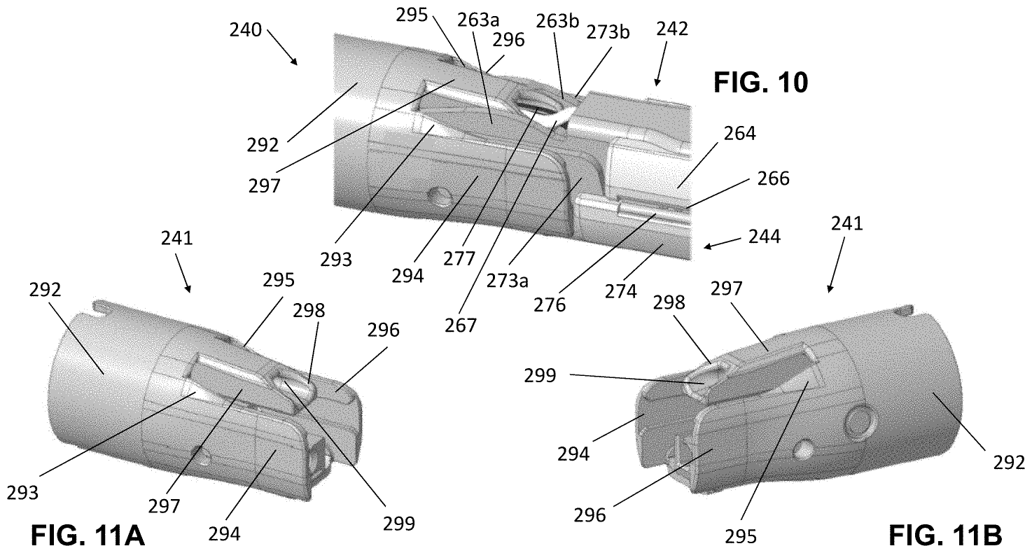

Referring to FIGS. 14A and 14B and FIGS. 15A and 15B, in conjunction with FIGS. 8-10, as noted above, second jaw member 244 is fixed relative to clevis 241, e.g., via welding proximal flags 273a, 273b on the interior sides of spaced-apart arms 294, 296, respectively. As such, in order to facilitate locating proximal flags 273a, 273b in proper alignment and orientation relative to respective spaced-apart arms 294, 296 and, thus, to facilitate securing proximal flags 273a, 273b to spaced-apart arms 294, 296, respectively, spaced-apart arms 294, 296 may include locating features 334, 336 (FIGS. 14A and 14B) or 434, 436 (FIGS. 15A and 15B) disposed on the interior sides thereof. More specifically, locating features 334, 336 (FIGS. 14A and 14B) or 434, 436 (FIGS. 15A and 15B) may be disposed at the proximal ends of spaced-apart arms 294, 296 and, in aspects, may be disposed underneath the overhangs defined by transitions 293, 295 (see also FIGS. 10-11B). Locating features 334, 336 (FIGS. 14A and 14B) or 434, 436 (FIGS. 15A and 15B) may be formed as protrusions, additional material, or additional components on the interior sides of spaced-apart arms 294, 296 and extending inwardly into the gap defined between arms 294, 296, although other configurations are also contemplated. Locating features 334, 336 (FIGS. 14A and 14B) or 434, 436 (FIGS. 15A and 15B) may include angled guide surfaces and/or stop surfaces to guide insertion of proximal flags 273a, 273b between spaced-apart arms 294, 296 and/or inhibit over-insertion of proximal flags 273a, 273b, respectively, thus facilitating proper positioning and alignment of proximal flags 273a, 273b. Even though proximal flags 273a, 273b of jaw member 244 are ultimately fixed relative to clevis 241, e.g., via welding, ensuring proper positioning and alignment thereof is important as the position of jaw member 244 affects the position and pivoting of jaw member 242 as well as the actuation of drive rod 1484 (FIG. 5) and the knife (not shown) relative to end effector assembly 240.

While several aspects of this disclosure have been shown in the drawings, it is not intended that this disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An end effector assembly of a surgical instrument, comprising:
a clevis defining a proximal body, first and second arms spaced-apart relative to one another and extending distally from the proximal body, and a finger extending distally from the proximal body at least partially between the first and second arms, the finger defining a mouth at a free distal end thereof and having a channel extending therethrough in communication with the mouth;
first and second jaw members supported by the clevis, each of the first and second jaw members including a proximal flange portion at least partially disposed between the first and second arms of the clevis, a distal body portion extending distally from the clevis, and an electrically conductive tissue contacting surface, at least one of the first or second jaw members movable relative to the other of the first or second jaw members and the clevis between a spaced-apart position and an approximated position for grasping tissue between the electrically conductive tissue contacting surfaces; and
first and second lead wires connected to the electrically conductive tissue contacting surfaces of the first and second jaw members, respectively, the first and second lead wires extending proximally from the distal body portions of the respective first and second jaw members through the mouth and into the channel of the finger of the clevis.

2. The end effector assembly according to claim 1, wherein the mouth is proximally-angled and defines a rounded edge.

3. The end effector assembly according to claim 1, wherein the proximal flange portions of the first and second jaw members are pivotably connected via a pivot pin disposed between the first and second arms of the clevis, and wherein at least one of the first or second lead wires extend proximally from the distal body portions of the respective first and second jaw members and traverses the pivot pin before entering the mouth.

4. The end effector assembly according to claim 1, wherein the proximal flange portions of the first and second jaw members each include first and second proximal flags spaced-apart relative to one another, wherein the first proximal flags of the proximal flange portions of the first and second jaw members are disposed on a first side of the finger, and wherein the second proximal flags of the proximal flange portions of the first and second jaw members are disposed on a second, opposite side of the finger.

5. The end effector assembly according to claim 4, wherein the proximal flange portions of the first and second jaw members define a nested configuration wherein the first and second proximal flags of the proximal flange portion of the first jaw member are disposed between the first and second proximal flags of the proximal flange portion of the second jaw member.

6. The end effector assembly according to claim 5, wherein the first and second proximal flags of the proximal flange portion of the second jaw member are fixed relative to the first and second arms of the clevis, respectively, to thereby fix the second jaw member relative to the clevis, and wherein the first jaw member is movable relative to the second jaw member and the clevis.

7. The end effector assembly according to claim 1, wherein the clevis includes a smoothed transition defining an internal overhang between each of the first and second arms and the proximal body.

8. The end effector assembly according to claim 1, wherein the proximal body and at least portions of lengths of the first and second arms taper in a proximal to distal direction such that the clevis defines a smooth taper in a lateral dimension in the proximal to distal direction.

9. The end effector assembly according to claim 8, wherein the clevis defines a maximum of the lateral dimension at the proximal end of the clevis, the maximum corresponding to a lateral dimension of a portion of a shaft jointed to the clevis.

10. The end effector assembly according to claim 8, wherein the clevis defines a minimum of the lateral dimension towards free ends of the first and second arms, the minimum corresponding to a maximum width of the distal body portions of the first and second jaw members.

11. An end effector assembly of a surgical instrument, comprising:
a clevis defining a proximal body, first and second arms spaced-apart relative to one another and extending distally from the proximal body, a floor extending distally from the proximal body between bottom edges of the first and second arms, and first and second cross supports connecting respective free end portions of the first and second arms with a free end portion of the floor, wherein the proximal body, the floor, the first and second arms, and the first and second cross supports cooperate to define and fully surround first and second longitudinally-extending slots disposed between the first arm and the floor and between the second arm and the floor, respectively; and
first and second jaw members supported by the clevis, each of the first and second jaw members including a proximal flange portion at least partially disposed between the first and second arms of the clevis, a distal body portion extending distally from the clevis, and an electrically conductive tissue contacting surface, the first jaw member movable relative to the second jaw member and the clevis between a spaced-apart position and an approximated position for grasping tissue between the electrically conductive tissue contacting surfaces,
wherein the proximal flange portion of the first jaw member includes first and second proximal flags spaced-apart relative to one another, wherein the first and second proximal flags extend through the first and second slots and externally of the clevis in at least the spaced-apart position of the first jaw member.

12. The end effector assembly according to claim 11, wherein the proximal flange portion of the second jaw member includes first and second proximal flags spaced-apart relative to one another and fixed relative to the first and second arms, respectively, of the clevis.

13. The end effector assembly according to claim 12, wherein the first and second proximal flags of the first jaw member are disposed between the first and second proximal flags of the second jaw member.

14. The end effector assembly according to claim 12, wherein the first and second arms define locating features on interior sides thereof to facilitate locating and fixing the first and second proximal flags of the second jaw member relative to the first and second arms, respectively.

15. The end effector assembly according to claim 11, wherein a maximum width of each of the first and second slots is within 15% of a maximum thickness of the first and second proximal flags.

16. The end effector assembly according to claim 11, wherein a maximum width of each of the first and second slots is within 10% of a maximum thickness of the first and second proximal flags.

17. The end effector assembly according to claim 11, wherein the proximal body and at least portions of lengths of the first and second arms taper in a proximal to distal direction such that the clevis defines a smooth taper in a lateral dimension in the proximal to distal direction.

18. The end effector assembly according to claim 17, wherein the clevis defines a maximum of the lateral dimension at the proximal end of the clevis, the maximum corresponding to a lateral dimension of a portion of a shaft jointed to the clevis.

19. The end effector assembly according to claim 17, wherein the clevis defines a minimum of the lateral dimension towards free ends of the first and second arms, the minimum corresponding to a maximum width of the distal body portions of the first and second jaw members.

20. The end effector assembly according to claim 11, wherein the clevis includes a smoothed transition defining an internal overhang between each of the first and second arms and the proximal body at upper edges of the first and second arms.
